(19) United States
(12) Patent Application Publication
(10) Pub. No.: US 2014/0005551 A1
(43) Pub. Date: Jan. 2, 2014

(54) ULTRASOUND PROBE AND ULTRASOUND DIAGNOSIS APPARATUS

(71) Applicant: Panasonic Corporation, Osaka (JP)

(72) Inventors: Masashi OZAWA, Kanagawa (JP); Koetsu SAITO, Tokyo (JP)

(73) Assignee: Panasonic Corporation, Osaka (JP)

(21) Appl. No.: 13/962,654

(22) Filed: Aug. 8, 2013

Related U.S. Application Data
(63) Continuation of application No. PCT/JP2012/005348, filed on Aug. 27, 2012.

(30) Foreign Application Priority Data
Aug. 31, 2011 (JP) 2011-189278

(51) Int. Cl. A61B 8/00

(52) U.S. Cl. CPC A61B 8/4444
USPC 600/459

(57) ABSTRACT

An ultrasound probe includes a piezoelectric material, a signal electrode provided on the piezoelectric material, and a ground electrode provided on the piezoelectric material. Ultrasound waves are emitted from the piezoelectric material by applying a voltage between the signal electrode and the ground electrode. A switching element switches a connection between the signal electrode and the ground electrode from a closed state to an open state, and vice versa, in accordance with a control signal for controlling whether or not to emit the ultrasound waves. The switching element changes from the closed state to the open state in accordance with the control signal for emitting the ultrasound waves, and changes from the open state to the closed state in accordance with the control signal for suspending emission of the ultrasound waves. The ultrasound probe can maintain the reliability of the piezoelectric material when the temperature is high or changes suddenly.

### BACKGROUND OF THE INVENTION

1. Field of the Invention

The present invention relates to an ultrasound probe that emits ultrasound waves into the body and receives the ultrasound waves reflected by tissues in the body, and an ultrasound diagnosis apparatus that includes the ultrasound probe.

2. Description of Related Art

Ultrasonography is a diagnostic imaging technique that produces images (ultrasound images) of tissues in the body for use in diagnosis. In the ultrasonography, ultrasound waves are directed into the body, and the ultrasound waves reflected by tissues in the body are received. Then, the images of the tissues in the body are produced and displayed. Specifically, to produce the ultrasound images, an ultrasound probe is placed in contact with the part of the body to be scanned, emits ultrasound waves into the body, receives ultrasound signals reflected by tissues in the body, and converts the ultrasound signals into electric signals.

The ultrasound probe is used only to examine a patient who needs an ultrasound imaging diagnosis. Thus, there is no problem with the ultrasound probe as long as it can generate ultrasound waves only when an ultrasound examination is performed. Moreover, the ultrasound probe should be stopped from emitting ultrasound waves at the time other than during the ultrasound examination, since the ultrasound wave emission may be a problem in terms of safety.

The ultrasound probe used for the ultrasonography includes a piezoelectric material to convert the ultrasound signals into the electric signals, and vice versa. In the ultrasonography, it is important for the piezoelectric material not only to have appropriate piezoelectric properties, but also to be less susceptible to the surrounding environment. The ultrasonography is applied to the body, and therefore is not performed in an environment where the temperature is too high for the body to endure or where the temperature changes suddenly. Accordingly, the ultrasound probe is operated generally at room temperature of about 20° C. However, the ultrasound probe may be exposed to a higher temperature than room temperature or a sudden temperature change when it is not used for the ultrasonography, e.g., during transportation or storage.

The piezoelectric material is a material that generates positive and negative charges on its surface when stress is applied to the material. It is known that the piezoelectric material has a direct piezoelectric effect (positive piezoelectric effect), in which polarization is generated by the application of stress, or an inverse piezoelectric effect, in which a distortion occurs in accordance with the magnitude of polarization. Moreover, it is also known that the piezoelectric material exhibits pyroelectricity such that spontaneous polarization in the piezoelectric material is changed due to temperature changes. As the temperature increases, the spontaneous polarization decreases and the amount of charge corresponding to the decrease in the spontaneous polarization is generated on the surface of the piezoelectric material. For example, a pyroelectric infrared sensor detects a change in the charge (i.e., a pyroelectric current) that is generated due to temperature changes when the piezoelectric material receives infrared radiation. Thus, the pyroelectric infrared sensor utilizes the pyroelectricity effectively. However, in the case of the ultrasound probe used for the ultrasonography, such a change in the charge on the surface of the piezoelectric material caused by the pyroelectricity may reduce the sensitivity characteristics of the ultrasound probe. If the sensitivity is reduced, the S/N of the ultrasound images of the inside of the body becomes low, and the necessary information cannot be imaged. Consequently, the patient's condition may be diagnosed incorrectly or cannot even be diagnosed, which leads to serious problems.

The piezoelectric material has a phase transition temperature called a Curie temperature. The piezoelectric material loses the piezoelectric properties at the Curie temperature or higher. It is recommended that the piezoelectric material be used generally at a temperature that is less than half the Curie temperature. In recent years, a solid solution single crystal composed of lead zinc niobate and lead titanate, a solid solution single crystal composed of lead magnesium niobate and lead titanate, or the like has attracted attention as a piezoelectric material having high piezoelectric properties. These solid solution single crystals have a phase transition temperature that is even lower than the Curie temperature. Therefore, the piezoelectric properties are likely to be degraded in an environment where the temperature is high or changes suddenly.

In piezoelectric applications, including the ultrasound probe used for the ultrasonography, a method for connecting a parallel resistance to the piezoelectric material has been known conventionally to prevent the degradation of the piezoelectric properties of the piezoelectric material due to a high temperature or a sudden temperature change (e.g., see JP S56 (1981)-144624 A 1 and JP S61 (1986)-94730 U).

FIG. 7 is a circuit diagram showing the configuration of a piezoelectric element 101 in which a resistor 103 is connected in parallel to a piezoelectric material 102. The piezoelectric material 102 has a signal electrode 104 on one side and a ground electrode 105 on the other side. The piezoelectric material 102 is deformed in accordance with the application of an external electric field by the inverse piezoelectric effect. The resistor 103 is connected to both the signal electrode 104 and the ground electrode 105. The resistor 103 is formed by baking a conductive paste on the signal electrode 104 and the ground electrode 105, or by providing a fixed resistor. With this configuration, a pyroelectric current flows from one electrode to the other electrode of the piezoelectric material 102 so as to suppress a change in the spontaneous polarization due to temperature changes, and thus the degradation of the piezoelectric properties can be prevented.

However, in the above conventional configuration, the resistor is connected to the signal electrode and the ground electrode of the piezoelectric material by using the conductive paste. Therefore, it is very difficult to apply the conductive paste to each of the several tens to several thousands of piezoelectric materials constituting the ultrasound probe so that the resistance values are uniform. On the other hand, providing the fixed resistors increases the number of circuit components. Therefore, it is very difficult to include the fixed resistors, particularly in the ultrasound probe that includes several thousands of piezoelectric materials.

Moreover, the individual piezoelectric materials differ in their optimum parallel resistance values. Thus, the optimum parallel resistance value needs to be found for each of the piezoelectric materials. In particular, when the ultrasound probe includes several tens to several thousands of piezoelectric materials, and the parallel resistances having the same value are connected to the piezoelectric materials, there is a risk that variations in the piezoelectric properties between the piezoelectric materials will be increased further.

The parallel resistances inserted into the piezoelectric materials can reduce the sensitivity of the ultrasound probe used for the ultrasonography. On the other hand, the rate of change in the piezoelectric properties due to temperature changes also varies according to the parallel resistance value. Here, a thermal shock test was performed on an ultrasound probe including a conventional piezoelectric element that uses a solid solution single crystal composed of lead magnesium niobate and lead titanate as a piezoelectric material, and has the same configuration as that shown in FIG. 7. FIG. 8 shows the Vpp sensitivity characteristics and the rate of change in the Vpp sensitivity before and after the thermal shock test with respect to the parallel resistance value.

The Vpp sensitivity characteristics are represented by the peak-to-peak value of the waveform of the electric signals that have been converted from the received ultrasound signals when the parallel resistance value is changed. In FIG. 8, the Vpp sensitivity is expressed as 100% without the parallel resistance. The rate of change in the Vpp sensitivity is obtained by (Vpp sensitivity after the test−Vpp sensitivity before the test)/(Vpp sensitivity before the test)×100 [%] when the parallel resistance value is changed. The thermal shock test was performed in the following manner. The piezoelectric element was allowed to stand at −20° C. for 1 hour and then allowed to stand at 60° C. for 1 hour. This process was defined as one cycle, and the cycle was repeated 100 times.

The Vpp relative sensitivity measured when the parallel resistance is connected before the thermal shock test increases with the parallel resistance value. The Vpp relative sensitivity is saturated and gets close to the sensitivity without the parallel resistance (parallel resistance value=∞) when the parallel resistance value reaches a certain level or higher. On the other hand, the rate of change in the Vpp sensitivity before and after the thermal shock test is saturated when the parallel resistance value reaches a certain level or higher, but increases as the parallel resistance value increases.

In other words, the Vpp sensitivity can remain high if a large parallel resistance value is selected. However, when the ultrasound probe is exposed to an environment similar to the thermal shock test, the Vpp sensitivity is reduced significantly and has an adverse effect on the ultrasonography. On the other hand, a reduction in the Vpp sensitivity in the environment of the thermal shock test can be suppressed if a small parallel resistance value is selected. In this case, however, the Vpp sensitivity is still low and has an adverse effect on the ultrasonography.

Therefore, there is a trade-off relationship between the piezoelectric properties and the reliability of the piezoelectric material in an environment where the temperature is high or changes suddenly. At present, the piezoelectric material is used by balancing the piezoelectric properties and the reliability or by sacrificing one of the piezoelectric properties and the reliability. In recent years, a piezoelectric material with a very high relative dielectric constant and a piezoelectric material with a very high piezoelectric constant such as a solid solution single crystal of lead zinc niobate and lead titanate or a solid solution single crystal of lead magnesium niobate and lead titanate have been put on the market. However, it is difficult to use these piezoelectric materials because the piezoelectric performance is reduced significantly by temperature changes. It is also difficult to make the best use of the piezoelectric performance of these piezoelectric materials, since the parallel resistance is connected and the piezoelectric properties are degraded intentionally so as to maintain the reliability.

The present invention solves the above conventional problems and has an object of providing an ultrasound probe and an ultrasound diagnosis apparatus that can prevent the degradation of piezoelectric properties of a piezoelectric material during operation for ultrasonography, and that can maintain the reliability of the piezoelectric material in an environment where the temperature is high or changes suddenly during non-operation (i.e., at the time other than the ultrasonography).

### SUMMARY OF THE INVENTION

To solve the above problems, an ultrasound probe of the present invention includes a piezoelectric material, a signal electrode provided on the piezoelectric material, and a ground electrode provided on the piezoelectric material, thereby being configured so that ultrasound waves are emitted from the piezoelectric material by applying a voltage between the signal electrode and the ground electrode. The ultrasound probe further includes a switching element that switches a connection between the signal electrode and the ground electrode from a closed state to an open state, and vice versa, in accordance with a control signal for controlling whether or not to emit the ultrasound waves.

The switching element may change from the closed state to the open state in accordance with the control signal for emitting the ultrasound waves, and may change from the open state to the closed state in accordance with the control signal for suspending emission of the ultrasound waves.

The switching element may operate to be the closed state in accordance with the control signal for suspending emission of the ultrasound waves.

The switching element may be a multiplexer.

The ultrasound probe may include a signal line connected to the signal electrode, and a ground line connected to the ground electrode. The switching element may include a contact plate that can be moved between a position where a connection between the signal line and the ground line is closed and a position where the connection between the signal line and the ground line is opened in accordance with the control signal.

The switching element may move the contact plate so that the connection between the signal line and the ground line is opened in accordance with the control signal for emitting the ultrasound waves, and may move the contact plate so that the connection between the signal line and the ground line is closed by the contact plate in accordance with the control signal for suspending emission of the ultrasound waves.

The switching element may move the contact plate to a position where the connection between the signal line and the ground line is closed in accordance with the control signal for suspending emission of the ultrasound waves, thereby providing the closed state.

The ultrasound probe further may include a detachable closing connector that closes the connection between the signal electrode and the ground electrode.

The ultrasound probe further may include a control unit that supplies the control signal to the switching element.

To solve the above problems, an ultrasound diagnosis apparatus of the present invention includes the above ultrasound probe and an ultrasound diagnosis apparatus main unit including a control unit that supplies the control signal to the switching element.

According to the ultrasound probe of the present invention, the switching element operates to be the open state during the emission of the ultrasound waves (during operation), and operates to be the closed state during the suspension of the emission of the ultrasound waves (during non-operation). Therefore, the ultrasound probe of the present invention can maintain the reliability of the piezoelectric material in an environment where the temperature is high or changes suddenly without degrading the piezoelectric properties.

### BRIEF DESCRIPTION OF THE DRAWINGS

FIG. 1 is a circuit diagram showing the schematic configuration of an ultrasound diagnosis apparatus of Embodiment 1 of the present invention.

FIG. 2 is a diagram showing the schematic configuration of a multiplexer.

FIG. 3A is a circuit diagram showing the schematic configuration of an ultrasound diagnosis apparatus of Embodiment 2 of the present invention when an ultrasound probe is operated.

FIG. 3B is a circuit diagram showing the schematic configuration of an ultrasound diagnosis apparatus of Embodiment 2 of the present invention when an ultrasound probe is not operated.

FIG. 3C is a circuit diagram showing the schematic configuration of an ultrasound diagnosis apparatus of Embodiment 2 of the present invention when an ultrasound probe is removed.

FIG. 4A is a side view showing the schematic configuration of an ultrasound probe of an ultrasound diagnosis apparatus of Embodiment 3 of the present invention, and illustrates a closed state.

FIG. 4B is a side view of the ultrasound probe when it is viewed from the direction of the arrow A in FIG. 4A.

FIG. 5A is a side view showing the schematic configuration of an ultrasound probe of an ultrasound diagnosis apparatus of Embodiment 3 of the present invention, and illustrates an open state.

FIG. 5B is a side view of the ultrasound probe when it is viewed from the direction of the arrow A in FIG. 5A.

FIG. 6 is a circuit diagram showing a state in which a closing connector is attached to an ultrasound probe of an ultrasound diagnosis apparatus of Embodiment 3 of the present invention.

FIG. 7 is a circuit diagram showing the configuration of a conventional piezoelectric element.

FIG. 8 is a graph showing the Vpp sensitivity characteristics of a piezoelectric material and a rate of change in the Vpp sensitivity before and after a thermal shock test.

### DETAILED DESCRIPTION OF THE INVENTION

Hereinafter, embodiments of an ultrasound diagnosis apparatus of the present invention will be described in detail with reference to the drawings.

### Embodiment 1

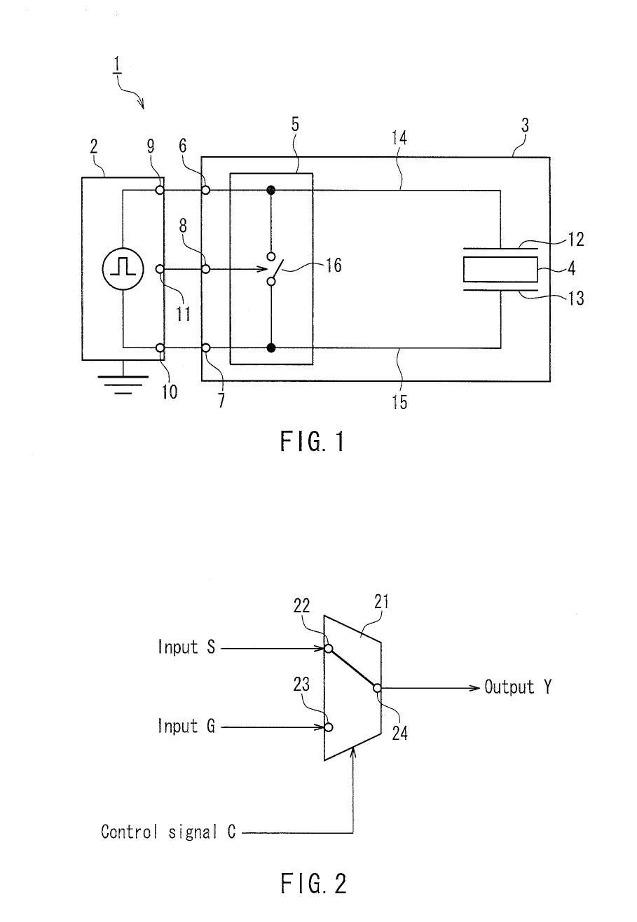

FIG. 1 is a circuit diagram showing the schematic configuration of an ultrasound diagnosis apparatus of Embodiment 1 of the present invention. An ultrasound diagnosis apparatus 1 includes an ultrasound diagnosis apparatus main unit 2 and an ultrasound probe 3.

The ultrasound diagnosis apparatus main unit 2 supplies a drive signal to the ultrasound probe 3 for driving the ultrasound probe 3 to emit an ultrasound signal, receives a received signal converted by the ultrasound probe 3 from the reflected ultrasound signal, and performs signal processing so that images (ultrasound images) of tissues in the body are displayed on a monitor (not shown). A main unit signal line terminal 9 supplies the drive signal and receives the received signal. A main unit ground line terminal 10 shows a ground potential. A main unit control line terminal 11 outputs a signal for controlling whether the ultrasound probe 3 is operated or not.

The ultrasound probe 3 is connected to the ultrasound diagnosis apparatus main unit 2 and used in contact with a subject. A piezoelectric material 4 converts electricity into ultrasound waves, and vice versa. Specifically, the piezoelectric material 4 is driven by the drive signal to emit an ultrasound signal, receives the ultrasound signal reflected by the subject, and converts the reflected ultrasound signal into a received signal. The capability to convert electricity into ultrasound waves or to convert ultrasound waves into electricity is determined by a piezoelectric d constant or piezoelectric g constant.

The relative dielectric constant of the piezoelectric material 4 relates to the electrical impedance of the piezoelectric material 4. The ultrasound probe 3 is provided with a large number of strip-shaped or columnar piezoelectric materials 4. Thus, the individual piezoelectric materials 4 decrease in size and increase in electrical impedance. Moreover, in recent years, an ultrasound probe 3 including a large number of smaller piezoelectric materials 4, which is called 1.5D array or matrix array, has appeared on the market. In this case, the electrical impedance of each of the piezoelectric materials 4 is increased further, and it is difficult to drive those piezoelectric materials 4 electrically. Therefore, a piezoelectric substance having a high piezoelectric constant or a high relative dielectric constant often is required for the piezoelectric material 4 of the ultrasound probe 3. However, the piezoelectric substance having a high piezoelectric constant or a high relative dielectric constant generally has a low Curie temperature and is likely to have reduced piezoelectric constant or relative dielectric constant due to temperature changes.

Examples of the substances for the piezoelectric material 4 include the following: piezoelectric ceramics of lead zirconate titanate or lead titanate; relaxor ferroelectrics having a very high relative dielectric constant; non-lead piezoelectric ceramics or piezoelectric single crystals of niobium, bismuth, or the like; a solid solution single crystal or ceramic of lead zinc niobate and lead titanate; a solid solution single crystal or ceramic of lead magnesium niobate and lead titanate; a solid solution single crystal or ceramic of lead indium niobate, lead magnesium niobate, and lead titanate; a solid solution single crystal or ceramic of lead magnesium niobate and lead zirconate titanate; and piezoelectric polymer membranes of PVDF (polyvinylidene fluoride) or the like.

The piezoelectric material 4 has a signal electrode 12 on one side and a ground electrode 13 on the other side. The signal electrode 12 and the ground electrode 13 are metallic materials such as gold or silver, and are formed for example by plating, sputtering, or baking. The signal electrode 12 is connected to a probe signal line terminal 6 via a signal line 14. The ground electrode 13 is connected to a probe ground line terminal 7 via a ground line 15.

The probe signal line terminal 6 can be connected to the main unit signal line terminal 9. The probe ground line terminal 7 can be connected to the main unit ground line terminal 10. The drive signals applied to the probe signal line terminal 6 and the probe ground line terminal 7 are supplied to the piezoelectric material 4 through the signal electrode 12 and the ground electrode 13. Moreover, the received signals produced by the piezoelectric material 4 are transmitted to the ultrasound diagnosis apparatus main unit 2 through the probe signal line terminal 6 and the probe ground line terminal 7.

A probe control line terminal 8 can be connected to the main unit control line terminal 11. A switching element 5 closes (short-circuits) or opens the connection between the signal line 14 and the ground line 15, i.e., the connection between the signal electrode 12 and the ground electrode 13. Specifically, the switching element 5 includes a switch 16 that is connected to the signal line 14 and the ground line 15, and is turned on and off by a control signal input from the probe control line terminal 8. That is, the switch 16 is turned off when the ultrasound probe 3 is operated (i.e., the ultrasound waves are emitted), and turned on when the ultrasound probe 3 is not operated (i.e., the ultrasound waves are not emitted).

In general, a plurality of probe signal line terminals 6 and a plurality of probe ground line terminals 7 are provided in accordance with the number of piezoelectric materials 4 and the specification of the ultrasound diagnosis apparatus main unit 2. For the sake of simplification, FIG. 1 shows one each of the probe signal line terminal 6 and the probe ground line terminal 7. However, the same configuration can be achieved even with the use of a plurality of terminals, except that the number of terminals is increased. In FIG. 1, the ultrasound diagnosis apparatus main unit 2 generates the drive signals and processes the received signals. In recent years, circuit boards for these signal processes are becoming smaller and smaller. Therefore, the above operations may be performed in the ultrasound probe 3.

One or more ultrasound probes 3 can be connected to the ultrasound diagnosis apparatus main unit 2, and a user can select any of the ultrasound probes 3 in accordance with the part of the body to be scanned. However, a plurality of ultrasound probes 3 cannot be used simultaneously. The electric signals are transmitted and received between the ultrasound diagnosis apparatus main unit 2 and the ultrasound probe 3 only when the ultrasound probe 3 is operated. No electric signal is transmitted and received between the ultrasound diagnosis apparatus main unit 2 and the ultrasound probe 3 when the ultrasound probe 3 is not operated.

The control signal is generated in accordance with the operation or non-operation of the ultrasound probe 3. The control signal is generated in the ultrasound diagnosis apparatus main unit 2 and transmitted through the main unit control line terminal 11 to the probe control line terminal 8 of the ultrasound probe 3. The control signal turns off the switch 16 during the operation of the ultrasound probe 3 and turns on the switch 16 during the non-operation of the ultrasound probe 3.

The control signal generated for the operation of the ultrasound probe 3 can be a signal that is generated when a user selects one of the ultrasound probes 3 connected to the ultrasound diagnosis apparatus main unit 2. If only one ultrasound probe 3 is connected to the ultrasound diagnosis apparatus main unit 2, although the user does not have to select the ultrasound probe 3, the connection of the ultrasound probe 3 is always confirmed before transmitting the electric signal to the ultrasound probe 3 to ensure the safety of the subject. Therefore, a signal that is generated when the connection is confirmed may be used as the control signal. In addition, any signal can be used as the control signal for the operation of the ultrasound probe 3 as long as the signal is generated immediately before the ultrasound probe 3 is operated.

The ultrasound probe 3 is not operated, e.g., in the following cases: the ultrasound probe 3 is not selected; the examination is stopped temporarily; the ultrasound diagnosis apparatus main unit 2 is turned off, the ultrasound probe 3 is removed from the ultrasound diagnosis apparatus main unit 2; or the ultrasound probe 3 is not connected to the ultrasound diagnosis apparatus main unit 2. A ground wire (not shown) of the ultrasound diagnosis apparatus main unit 2 can be used for the control signal to be generated when the ultrasound probe 3 is not selected. The ultrasound diagnosis apparatus main unit 2 has the function of stopping the examination temporarily without removing the ultrasound probe 3 or cutting off the power supply. Therefore, a signal that is generated when this function is performed may be used as the control signal. Moreover, a signal that is generated when the ultrasound diagnosis apparatus main unit 2 is turned off also may be used as the control signal.

Since the electric signal at a very high voltage is applied from the ultrasound diagnosis apparatus main unit 2 to the ultrasound probe 3 during the operation, if the ultrasound probe 3 is removed from the ultrasound diagnosis apparatus main unit 2 in this state, the circuit (not shown) in the ultrasound probe 3 can be damaged. Therefore, the ultrasound probe 3 usually is removed after the ultrasound diagnosis apparatus main unit 2 is turned off or brought to a state in which the examination is stopped temporarily. Thus, at the time of the removal of the ultrasound probe 3, a signal that is generated when the power supply is cut off or when the examination is stopped temporarily may be used as the control signal. In addition, any signal can be used as the control signal for the non-operation of the ultrasound probe 3 as long as the signal is generated immediately before the operation of the ultrasound probe 3 is stopped or interrupted.

In this embodiment, the control signal is transmitted to the ultrasound probe 3 by connecting the main unit control line terminal 11 to the probe control line terminal 8. However, the control signal may be transmitted wirelessly from the ultrasound diagnosis apparatus main unit 2 if the ultrasound diagnosis apparatus main unit 2 is provided with a radio transmitter (not shown) and the ultrasound probe 3 is provided with a radio receiver (not shown).

In FIG. 1, the control signal is generated in the ultrasound diagnosis apparatus main unit 2. However, the ultrasound probe 3 may include a means for generating the control signal. As the means for generating the control signal, e.g., an acceleration sensor (not shown) is provided in the ultrasound probe 3, and the output of the acceleration sensor may be used as the control signal. During the operation, a user holds the ultrasound probe 3 by hand and scans the surface of the body with the ultrasound probe 3. Therefore, it can be found from the output of the acceleration sensor that the ultrasound probe 3 is being operated. During the non-operation, the user puts the ultrasound probe 3 in a holder (not shown) that is attached to the ultrasound diagnosis apparatus main unit 2 or puts the ultrasound probe 3 away to prevent it from being touched by hand. Therefore, the output of the acceleration sensor is not changed, from which it can be found that the ultrasound probe 3 is not being operated. In other words, the control signal can be generated by utilizing a difference in the movement of the ultrasound probe 3 between the operation state and the non-operation state. Similarly, the output of a sensor other than the acceleration sensor may be used as the control signal by utilizing a difference in the movement of the ultrasound probe 3 between the operation state and the non-operation state.

As described above, in this embodiment, the connection between the signal electrode 12 and the ground electrode 13 is opened during the operation of the ultrasound probe 3, so that the ultrasound signals can be detected with high sensitivity.

On the other hand, the ultrasound probe 3 is not always maintained at room temperature and may be exposed to a higher temperature or a sudden temperature change when it is not operated, specifically during transportation or storage. However, the connection between the signal electrode 12 and the ground electrode 13 is closed, thereby suppressing a reduction in sensitivity.

Therefore, this embodiment can use a piezoelectric material that has high piezoelectric properties, but may significantly degrade the piezoelectric properties in an environment where the temperature is high or changes suddenly. Thus, an ultrasound diagnostic image can be produced with even higher sensitivity.

### Embodiment 2

An ultrasound diagnosis apparatus of Embodiment 2 of the present invention is characterized by the use of a multiplexer as the switching element 5. The other configurations are the same as those of the ultrasound diagnosis apparatus of Embodiment 1. The same components as those of the ultrasound diagnosis apparatus of Embodiment 1 are denoted by the same reference numerals, and the explanation will not be repeated.

A multiplexer 21 has a mechanism like a switch and outputs a signal with respect to a plurality of input signals. FIG. 2 is a diagram showing the schematic configuration of a simple two-input multiplexer. When two input signals S, G are input to input terminals 22, 23, respectively, an output signal Y expressed by the following logical expression (formula 1) is output from an output terminal 24 of the multiplexer 21 in accordance with the value of a control signal C.

Y=(S·C)+(G·C*) (Formula 1)

where * represents negative logic, and the control signal C is either “0” or “1”.

The above logical expression indicates selecting the input signal S or the input signal G. If the control signal C is “1”, then the output signal Y is “S” based on the formula 1. On the other hand, if the control signal C is “0”, then the output signal Y is “G” based on the formula 1. Thus, the multiplexer 21 is operated as a switch that connects one of the input terminals 22, 23 to the output terminal 24 in accordance with the control signal C.

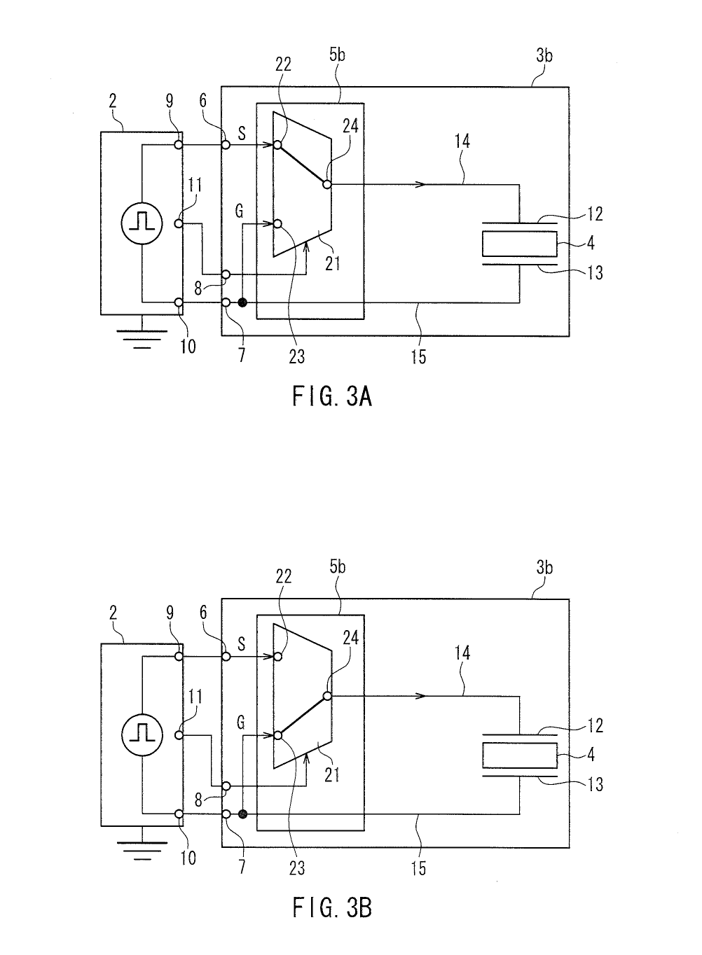

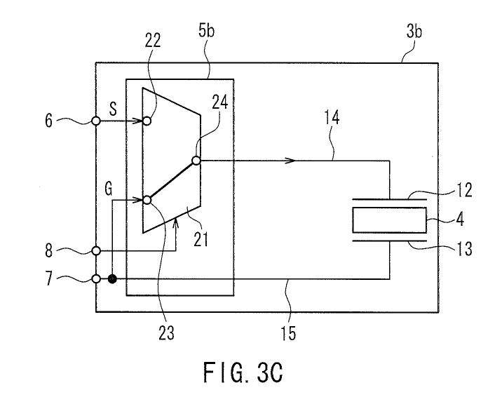

FIG. 3 is a schematic diagram showing the configuration of an ultrasound probe 3b in which the two-input multiplexer that is operated as described above is applied to the switching element 5. FIG. 3A shows a state in which the ultrasound probe 3b is operated (i.e., diagnostic imaging is performed). FIG. 3B shows a state in which the ultrasound probe 3b is not operated. FIG. 3C shows a state in which the ultrasound probe 3b is removed from the ultrasound diagnosis apparatus main unit 2.

The signal electrode 12 provided on the piezoelectric material 4 is connected to the output terminal 24 of the multiplexer 21 via the signal line 14. On the other hand, the ground electrode 13 is connected to the probe ground line terminal 7 via the ground line 15 and also connected to the input terminal 23 of the multiplexer 21. The probe signal line terminal 6 is connected to the input terminal 22 of the multiplexer 21. The probe control line terminal 8 is connected to an input terminal (not shown) of the control signal C.

As shown in FIG. 3A, if the control signal C input to the multiplexer 21 is “1”, the output signal Y is “S”. That is, the input terminal 22 is connected to the output terminal 24, and the connection between the signal line 14 and the ground line 15 is opened. Therefore, the drive signal is applied to the piezoelectric material 4, so that ultrasound waves can be emitted. Thus, the ultrasound signals can be detected with high sensitivity.

As shown in FIG. 3B, if the control signal C input to the multiplexer 21 is “0”, the output signal Y is “G”. That is, the input terminal 23 is connected to the output terminal 24, and the connection between the signal line 14 and the ground line 15 is closed. Therefore, no voltage is applied to the piezoelectric material 4, so that ultrasound waves cannot be emitted. Thus, even if the ultrasound probe 3b is exposed to a higher temperature or a sudden temperature change, the degradation of the piezoelectric properties can be suppressed.

As shown in FIG. 3C, when the ultrasound probe 3b is not connected to the ultrasound diagnosis apparatus main unit 2, the control signal C is set to “0”. That is, similarly to FIG. 3B, the connection between the signal line 14 and the ground line 15 is closed when the ultrasound probe 3b is not connected to the ultrasound diagnosis apparatus main unit 2. Thus, even if the ultrasound probe 3b is exposed to a higher temperature or a sudden temperature change, the degradation of the piezoelectric properties can be suppressed.

FIG. 3 shows an example in which one multiplexer 21 is used to switch the connection between the signal line 14 and the ground line 15, i.e., the connection between the signal electrode 12 and the ground electrode 13 from the open state to the closed state, and vice versa, with respect to one piezoelectric material 4. At present, there is an integrated-circuit multiplexer 21 that can provide switching of 16 outputs or 32 outputs. Therefore, even if the number of piezoelectric materials 4 is increased, the switching between the open state and the closed state can be performed only with a limited number of multiplexers 21, which are sufficiently small to be contained in the ultrasound probe 3b. Thus, the multiplexer 21 is suitable for the switching element 5 when the number of piezoelectric materials 4 is increased.

Moreover, commercially available switching devices (not shown) such as a relay circuit, a transistor, and a photocoupler also may be used as the switching element 5. Any of these switching devices is connected as the switching element 5 in the same manner as that shown in FIG. 3, and the control signal is transmitted to the coil, the base of the transistor, or the light emitting diode. With this configuration, the switching element 5 can be switched to the open state or the closed state by the control signal.

### Embodiment 3

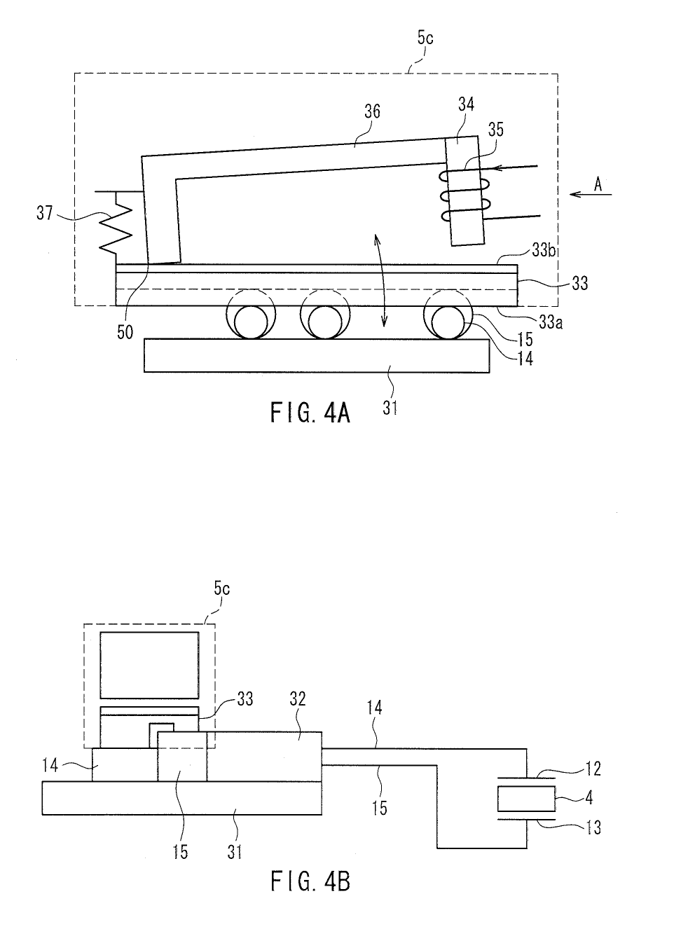

FIG. 4A is a side view showing the schematic configuration of an ultrasound probe of an ultrasound diagnosis apparatus of Embodiment 3 of the present invention. FIG. 4B is a side view of the ultrasound probe when it is viewed from the direction of the arrow A in FIG. 4A. In FIGS. 4A and 4B, the signal line 14 and the ground line 15 are illustrated schematically as coaxial lines. The ultrasound diagnosis apparatus of this embodiment uses a different configuration from that of Embodiment 2 as the switching element 5 of the ultrasound diagnosis apparatus of Embodiment 1. In the ultrasound diagnosis apparatus of this embodiment, the same components as those of the ultrasound diagnosis apparatus of Embodiment 1 are denoted by the same reference numerals, and the explanation will not be repeated.

The signal line 14 and the ground line 15 are connected to a coaxial cable 32, e.g., by soldering, a conductive paste, or jointing. The inner conductor of the coaxial cable 32 is used as the signal line 14, and the outer conductor of the coaxial cable 32 is used as the ground line 15. Instead of the coaxial cable, it is also possible to use a single-wire cable, a metal foil sheet, or a flexible conductive material such as a copper-clad laminate used for a flexible substrate. The signal line 14 and the ground line 15 of the coaxial cable 32 may be, e.g., arranged on a printed board 31 and connected by soldering or the like, and further connected to the probe signal line terminal 6 and the probe ground line terminal 7 (which are shown in FIG. 1) via connectors (not shown) provided on the printed board 31.

As shown in FIG. 4A, a switching element 5c includes a coil 35, a contact plate 33, an iron core 34, a magnetite 36, and a spring 37. The switching element 5c switches the connection between the signal line 14 and the ground line 15 from the open state to the closed state, and vice versa, by the electromagnetic force of the coil 35.

The contact plate 33 is in contact with the magnetite 36 and can be rotated around a contact 50 with the magnetite 36. Thus, the contact plate 33 is detachable from the signal line 14 and the ground line 15. Moreover, the contact plate 33 is configured so that a portion 33a that comes into contact with the signal line 14 and the ground line 15 is made of a conductive material, and a portion 33b that faces the iron core 34 is made of a magnetic material. Therefore, the rotation of the contact plate 33 allows the connection between the signal line 14 and the ground line 15 to be switched from the closed state to the open state, and vice versa. The shape and size of the contact plate 33 can be selected freely, e.g., in accordance with the size and arrangement of the signal line 14 and the ground line 15. For example, FIG. 4 shows the U-shaped contact plate 33 (see FIG. 4B) so as to close the connection between the signal line 14 and the ground line 15 that are connected on the printed board 31.

The magnetite 36 is formed into an L shape, and one end is connected to the iron core 34 and the other end is in contact with the contact plate 33. The coil 35 is wound around the iron core 34. One end of the spring 37 is attached to the side of the contact plate 33 that is opposite to the iron core 34 with respect to the contact 50 of the contact plate 33 with the magnetite 36, and the other end of the spring 37 is attached to the magnetite 36. In general, as shown in FIG. 4A, the contact plate 33 is separated from the iron core 34 due to the force of the spring 37 and is connected to the signal line 14 and the ground line 15. Thus, the connection between the signal line 14 and the ground line 15 is closed.

When the ultrasound probe 3 is not operated, the control signal does not flow through the coil 35, as shown in FIGS. 4A and 4B. In this state, the connection between the signal line 14 and the ground line 15 is closed. Therefore, an electric signal cannot be applied to the piezoelectric material 4, so that ultrasound waves cannot be emitted. Thus, even if the ultrasound probe 3 is exposed to a higher temperature or a sudden temperature change, the degradation of the piezoelectric properties can be suppressed.

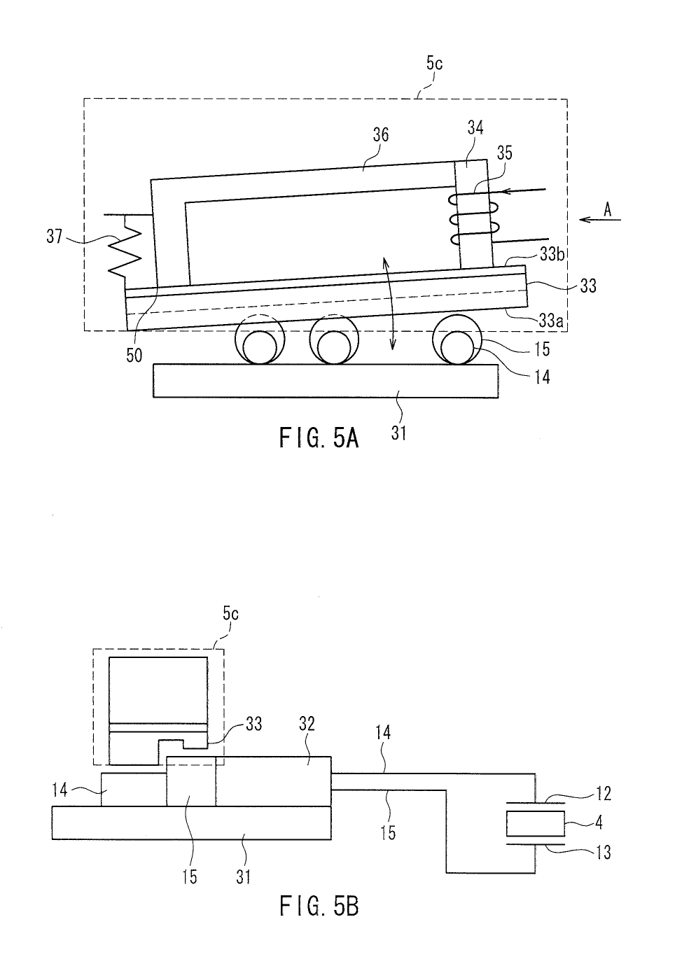

FIG. 5A is a side view showing the schematic configuration of the ultrasound probe of the ultrasound diagnosis apparatus during the operation, and illustrates a state in which the ultrasound probe is operated. FIG. 5B is a side view of the ultrasound probe when it is viewed from the direction of the arrow A in FIG. 5A. When the ultrasound probe 3 is operated, the control signal flows from the probe control line terminal 8 (see FIG. 1) to the coil 35. The current flowing through the coil 35 magnetizes the iron core 34. The magnetic flux generated in the iron core 34 passes through the magnetite 36 and the contact plate 33 (magnetic material), so that an electromagnetic force is produced to cause the portion of the contact plate 33 facing the iron core 34 to be attracted toward the iron core 34. This eliminates the contact between the contact plate 33 and the signal line 14 and the ground line 15, and consequently the connection between the signal line 14 and the ground line 15 is opened. That is, the connection between the signal electrode 12 and the ground electrode 13 is opened. Therefore, the drive signal is transmitted to the ultrasound probe 3 and applied to the piezoelectric material 4, so that ultrasound waves can be emitted. Thus, the ultrasound signals can be detected with high sensitivity.

When the ultrasound probe 3 is switched from the operation state to the non-operation state, the control signal is stopped from flowing through the coil 35. Then, the iron core 34 is not magnetized, and the contact plate 33 loses the electromagnetic force. Accordingly, due to the force of the spring 37, the contact plate 33 returns to the position where it is in contact with the signal line 14 and the ground line 15 (see FIGS. 4A and 4B). That is, the connection between the signal electrode 12 and the ground electrode 13 is restored to the closed state. Therefore, an electric signal cannot be applied to the piezoelectric material 4, so that ultrasound waves cannot be emitted. Thus, even if the ultrasound probe 3 is exposed to a higher temperature or a sudden temperature change, the degradation of the piezoelectric properties can be suppressed.

The contact plate 33 is moved to the position where the connection between the signal line 14 and the ground line 15 is opened or to the position where the connection between the signal line 14 and the ground line 15 is closed in accordance with the control signal generated during the operation or non-operation of the ultrasound probe 3. This configuration can switch the connection between the signal electrode 12 and the ground electrode 13 of the piezoelectric material 4 from the open state to the closed state, and vice versa.

In the ultrasound probe 3 having a plurality of piezoelectric materials 4, several switching elements 5c may be provided, thereby switching the connection between the signal line 14 and the ground line 15 from the open state to the closed state, and vice versa.

Moreover, the switching element 5c may have a configuration using a motor (not shown) and a wire (not shown). For example, the contact plate 33 and the motor are connected by the wire. When the ultrasound probe 3 is operated, the control signal in the operation state flows through the motor, and the wire is wound up so that the contact plate 33 is separated from the signal line 14 and the ground line 15. Thus, the connection between the signal line 14 and the ground line 15 is opened. On the other hand, when the ultrasound probe 3 is not operated, the control signal in the non-operation state flows through the motor, e.g., so as to drive the motor in reverse to the direction in which the motor rotates during the operation, and the wire is unwound until the contact plate 33 comes into contact with the signal line 14 and the ground line 15. Thus, the connection between the signal line 14 and the ground line 15 is closed. Even this method can switch the connection between the signal electrode 12 and the ground electrode 13 of the piezoelectric material 4 from the open state to the closed state, and vice versa.

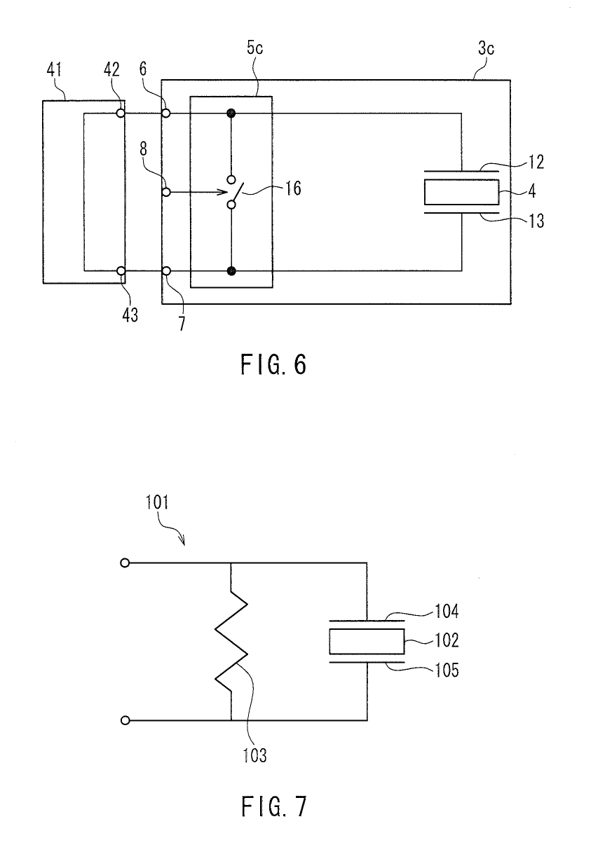

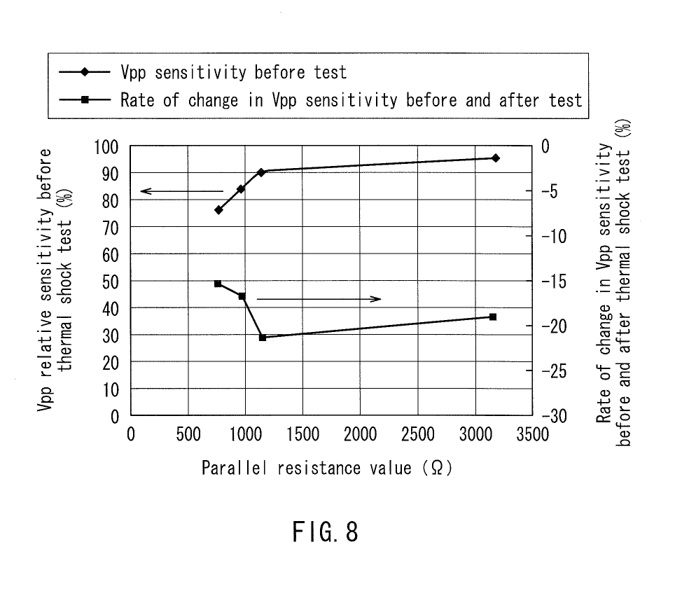

Transportation is an example of the non-operation of the ultrasound probe 3c. During the transportation, the ultrasound probe 3c can be subjected not only to a severe temperature environment, but also to a mechanical shock. In some cases, the closed state cannot be stabilized, particularly during the transportation, by the method that uses the switching element 5c and the contact plate 33 to switch the connection between the signal line 14 and the ground line 15 mechanically from the open state to the closed state, and vice versa. Therefore, a detachable closing connector 41 may be used, in which the terminals to be connected to the probe signal line terminal 6 and the probe ground line terminal 7 of the ultrasound probe 3c are closed in advance. FIG. 6 shows the configuration of the ultrasound probe 3c using the closing connector 41. The connection between a signal line terminal 42 and a ground line terminal 43 of the closing connector 41 is closed.

The closing connector 41 can be removed from the ultrasound probe 3c. The signal line terminal 42 can be connected to the probe signal line terminal 6 and the ground line terminal 43 can be connected to the probe ground line terminal 7. A connector (not shown) for connecting the ultrasound probe 3c to the ultrasound diagnosis apparatus main unit 2 has high resistance to mechanical shock and is suitable for a connector used for the closing connector 41.

In particular, when the ultrasound probe 3c is not operated, e.g., during the transportation, the closing connector 41 is connected to the ultrasound probe 3c. When the ultrasound probe 3c is operated, the closing connector 41 is removed from the ultrasound probe 3c. This method further can improve the reliability of the closed state between the signal electrode 12 and the ground electrode 13 of the piezoelectric material 4 during the transportation. The closing connector 41 may also be used for the ultrasound probe 3 including the electrical switching element 5 such as the multiplexer 21.

The present invention is useful as an ultrasound probe used for the ultrasonography, since the present invention utilizes the fact that the operating temperature is limited at about room temperature, and has the effect of being able to achieve both high piezoelectric properties and high reliability even with the use of a piezoelectric material that has high piezoelectric properties, but may suffer significantly degraded piezoelectric properties in an environment where the temperature is high or changes suddenly.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. An ultrasound probe comprising:
a piezoelectric material;
a signal electrode provided on the piezoelectric material; and
a ground electrode provided on the piezoelectric material,
thereby being configured so that ultrasound waves are emitted from the piezoelectric material by applying a voltage between the signal electrode and the ground electrode,
wherein the ultrasound probe further comprises a switching element that switches a connection between the signal electrode and the ground electrode from a closed state to an open state, and vice versa, in accordance with a control signal for controlling whether or not to emit the ultrasound waves.

2. The ultrasound probe according to claim 1, wherein the switching element changes from the closed state to the open state in accordance with the control signal for emitting the ultrasound waves, and changes from the open state to the closed state in accordance with the control signal for suspending emission of the ultrasound waves.

3. The ultrasound probe according to claim 1, wherein the switching element operates to be the closed state in accordance with the control signal for suspending emission of the ultrasound waves.

4. The ultrasound probe according to claim 1, wherein the switching element is a multiplexer.

5. The ultrasound probe according to claim 1, comprising:
a signal line connected to the signal electrode; and
a ground line connected to the ground electrode,
wherein the switching element includes a contact plate that can be moved between a position where a connection between the signal line and the ground line is closed and a position where the connection between the signal line and the ground line is opened in accordance with the control signal.

6. The ultrasound probe according to claim 5, wherein the switching element moves the contact plate so that the connection between the signal line and the ground line is opened in accordance with the control signal for emitting the ultrasound waves, and moves the contact plate so that the connection between the signal line and the ground line is closed by the contact plate in accordance with the control signal for suspending emission of the ultrasound waves.

7. The ultrasound probe according to claim 5, wherein the switching element moves the contact plate to a position where the connection between the signal line and the ground line is closed in accordance with the control signal for suspending emission of the ultrasound waves, thereby providing the closed state.

8. The ultrasound probe according to claim 1, further comprising a detachable closing connector that closes the connection between the signal electrode and the ground electrode.

9. The ultrasound probe according to claim 1, further comprising a control unit that supplies the control signal to the switching element.

10. An ultrasound diagnosis apparatus comprising:
the ultrasound probe according to claim 1; and
an ultrasound diagnosis apparatus main unit including a control unit that supplies the control signal to the switching element.
